(19) United States
(12) Patent Application Publication
(10) Pub. No.: US 2021/0268082 A1
(43) Pub. Date: Sep. 2, 2021

(54) METHOD OF INDUCING IMMUNE TOLERANCE BY ADMINISTERING ACTIVE PRINCIPLE-LOADING RBC's

(71) Applicant: Erytech Pharma, Lyon (FR)

(72) Inventors: Yann GODFRIN, Lyon (FR); Alice BANZ, Lyon (FR)

(21) Appl. No.: 17/169,357

(22) Filed: Feb. 5, 2021

Related U.S. Application Data
(63) Continuation of application No. 13/503,955, filed on Apr. 25, 2012, now abandoned.
(60) Provisional application No. 61/325,511, filed on Apr. 19, 2010.
(60) Provisional application No. 61/255,250, filed on Oct. 27, 2009.

(51) Int. Cl. A61K 39/00; A61K 35/18

(52) U.S. Cl. CPC A61K 39/0008; A61K 38/00; A61K 2039/5158; A61K 35/18

(57) ABSTRACT

The invention relates to a composition which induces, in a host, an immune tolerance to a peptidic or proteic active principle, said composition comprising red blood cells containing an active principle selected from the group consisting of a therapeutic peptide, polypeptide or protein, a peptidic or proteic autoantigen, peptide, polypeptide or protein inducing an allergic reaction and a transplantation peptidic or proteic antigen.

### CROSS-REFERENCE TO RELATED APPLICATIONS

This application is a continuation patent application of U.S. patent application Ser. No. 13/503,955, which is an International Application No. PCT/EP2010/066269, filed on Oct. 27, 2010 which claims benefit of U.S. Provisional Application No. 61/325,511, filed Apr. 19, 2010 and U.S. Provisional Application No. 61/255,250, filed on Oct. 27, 2009. The disclosures of these applications are hereby incorporated herein by reference in their entirety.

### BACKGROUND OF THE INVENTION

The present invention relates to a composition which induces, in a host, an immune tolerance to a peptidic or proteic active principle, in particular a therapeutic peptide, polypeptide or protein, a peptidic or proteic autoantigen, a peptide, polypeptide or protein inducing an allergic reaction or a transplantation peptidic or proteic antigen. The invention also relates to a method of treatment of a mammal, including human.

The liver is known to favour the induction of immune tolerance. This is exemplified by tolerization of food antigens in the liver and acceptance of liver allografts. There has been also some demonstration of antigen-specific tolerance to some foreign antigens delivered into the liver. E. Breous et al., Hepatology, August 2009, pp 612-621 report that hepatic regulatory T cells and Kupffer cells are crucial mediators of systemic T cell tolerance to antigens targeting murine liver. They report that in a model of liver-directed gene transfer, cytotoxic T lymphocyte responses to non-self antigens are controlled by hepatic regulatory T cells that secrete the immunosuppressive cytokine interleukin IL-10 in response to the antigen. In addition, the Kupffer cells are rendered tolerogenic rather than generating an immune response in this context.

The tolerogenic role of Kupffer cells has also been reported by C. Ju et al., Chem. Res. Toxicol. 2003, 16:1514-1519. See also A. H. Lau et al., Gut 2003, 52:1075-1078.

The present invention aims at providing compositions that can be used for the induction of an immune tolerance against a variety of peptidic or proteic active principles. It aims in particular at providing a specific immune tolerance with respect to one or several peptidic or proteic active principles.

An object of the invention is therefore a composition which induces, in a host, an immune tolerance to a peptidic or proteic active principle, said composition comprising red blood cells containing said active principle. This active principle may be a therapeutic peptide, polypeptide or protein, a peptidic or proteic autoantigen, a peptide, polypeptide or protein inducing an allergic reaction or a transplantation peptidic or proteic antigen, and mixtures thereof.

The active principle may be of natural, synthetic or recombinant origin. By “containing” molecule, it is intended to encompass molecules that contain the peptide, polypeptide or protein of interest and another moiety that may be of any origin and is not detrimental to the action of said peptide, polypeptide or protein. For example, such moiety includes haptens.

Without being bound to theory, the composition according to the invention is deemed to induce antigen(s)-specific regulatory T cells (Tregs) and to produce immunosuppressive cytokines or interleukins, in particular IL-10.

Biological and/or biotechnology-derived peptides, polypeptides or proteins are increasingly used as therapeutic agents. It has been however recognised that these agents may induce humoral and/or cellular immune responses. The consequences of an immune reaction to such therapeutic agent range from transient appearance of antibodies without any clinical significance to severe life threatening conditions. Potential clinical consequences are severe hypersensitivity-type reactions, decrease in efficacy and induction of auto-immunity, including antibodies to the endogenous form of the peptide, polypeptide or protein (European Medicines Agency, Committee for Medicinal products for human use (CHMP), Guidelines on immunogenicity assessment of biotechnology-derived therapeutic proteins, Draft, London, 24 Jan. 2007).

A therapeutic peptide, polypeptide or protein is by definition a peptide, polypeptide or protein or a peptide, polypeptide or protein containing molecule that is efficient in treating a pathology, especially a pathology due to a deficiency that can be corrected by administration of this molecule.

In an embodiment, the therapeutic peptide, polypeptide or protein is an antibody. This encompasses any fragment thereof.

In another embodiment, the therapeutic peptide, polypeptide or protein is a clotting factor. This encompasses any fragment thereof.

In another embodiment, the therapeutic peptide, polypeptide or protein is an enzyme. This encompasses any fragment thereof.

In another embodiment, the therapeutic peptide, polypeptide or protein is a growth factor. This encompasses any fragment thereof.

The term fragment is used to encompass any fragment of the peptide, polypeptide or protein that is known to be efficient in treating the associated pathology in replacement to the whole molecule.

The glycosylated forms are also encompassed by these definitions.

In an embodiment, the active principle is a lysosomal enzyme. The lysosomal enzyme may be one used to treat or correct a lysosomal storage disease by enzyme replacement therapy (ERT), including pompe disease (Glycogen storage disease type II), Fabry disease and Mucopolysaccharidoses disorders MPS I. As examples, one may mention:
alphaglucosidase enzyme, e.g. Myozyme® to treat pompe disease;
laronidase, e.g. Aldurazyme® to treat MPS I;
alphagalactosidase A or agalsidase alpha, e.g. Fabrazyme® and Replagal® to treat the Fabry disease.

In another embodiment, the active principle is a clotting factor useful in treating Haemophilia. The clotting factor may be Factor VIII, in particular for treating Haemophilia A. The clotting factor may be Factor IX, in particular for treating Haemophilia B. The clotting factor may be Factor VII for treating both Haemophilias.

A peptidic or proteic autoantigen is by definition an antigen that is a normal tissue constituent and is in a patient the target of a detrimental humoral or cell-mediated immune response, as in autoimmune disease.

In an embodiment, the active principle is against Rheumatoid Arthritis (RA).

In another embodiment, the active principle is against Multiple Sclerosis (MS). For example the active principle is myelin basic protein.

In another embodiment, the active principle is against Juvenile diabetes, such as diabetes type 1 and LADA (Latent Autoimmune Diabetes of Adults). As examples, one may cite the Beta-cell antigen, in particular glutamic acid decarboxylase (GAD), the pro-insulin and the insulin-like growth factor-2 (IGF2), and mixtures thereof.

In another embodiment, the active principle is against Uveitis. One may cite the retinal-S antigen.

In another embodiment, the active principle is against an inflammatory bowel disease (IBD), such as Crohn's disease and ulcerative colitis.

In another embodiment, the active principle is against systemic lupus erythematosus.

In another embodiment, the active principle is against psoriasis.

In another embodiment, the active principle is against acquired myasthenia gravis. As example, one may cite the acetyl choline receptor.

A peptide, polypeptide or protein inducing an allergic reaction is by definition a peptide, polypeptide or protein which is responsible for an allergic reaction in a host which reaction may include anaphylactic chock.

In an embodiment, this peptide, polypeptide or protein inducing an allergic reaction is a therapeutic active peptide, polypeptide or protein as mentioned above, wherein the present invention allows avoiding some or any allergic reaction against it and neutralization thereof.

In another embodiment, the peptide, polypeptide or protein inducing an allergic reaction is of food origin or any other proteic or peptidic molecule that may enter the blood circulation and create allergic reaction, e.g. after oral ingestion.

A transplantation peptidic or proteic antigen is by definition an antigen that is presented by the transplanted tissue and is involved in the patient in the graft rejection, say Graft Versus Host Disease (GVHD).

In an embodiment, the transplantation antigen is one involved in kidney graft rejection.

In an embodiment, the transplantation antigen is one involved in heart graft rejection.

In an embodiment, the transplantation antigen is one involved in liver graft rejection.

The term “host” refers preferably to humans, but also to animals, in particular pets (especially dogs or cats) and animals for sport (especially horses).

According to the invention, the red blood cells contain, i.e. encapsulate, the active principle (AP), which means that the AP is or is essentially inside the red blood cells.

In an embodiment, the composition targets the antigen-presenting cells (APCs) of the reticuloendothelial system. According to a feature, the red blood cells are designed, selected or modified so as to promote targeting of the antigen-presenting cells (APCs) of the reticuloendothelial system.

In a preferred embodiment, the composition targets the liver and especially the Kupffer cells. According to a feature, the red blood cells are designed, selected or modified so as to promote targeting of the liver. Delivering the AP to the liver results in the induction of AP tolerance and especially AP-specific tolerance. The liver's tolerogenic APCs are implicated in the induction of this tolerance. These cells are essentially Kupffer cells (KCs), non mature hepatic dendritic cells and liver sinusoidal endothelial cells.

In a preferred embodiment, the composition is used to repress the proinflammatory response of APCs. According to a feature, the red blood cells are preferably designed or modified so as to repress the proinflammatory response of APCs.

In a preferred embodiment, the compositions according to the invention comprise red blood cells which contain the AP and target the liver. The composition promotes phagocytosis of these red blood cells by the liver's APCs, especially the KCs.

According to a first embodiment, the red blood cells contain the AP and are in the form of an immune complex with an immunoglobulin which recognizes an epitope at the surface of said red blood cells, so as to promote the phagocytosis of said red blood cells by the liver's APCs, especially the KC.

The composition also makes it possible to promote phagocytosis by macrophages.

Preferably, the immunoglobulin is an immunoglobulin G.

As antibody that may be used to make adequate opsonisation, one may mention anti-Rhesus antibodies, anti-glycophorine A antibodies and anti-CR1 (CR1=type 1 complement receptor) antibodies. Anti-Rhesus antibodies are preferred.

According to another embodiment, the liver targeting and/or the inhibition of the proinflammatory response is/are done by an appropriate chemical treatment using agents which modify the surface of red blood cells, and in particular bridging or crosslinking agents such as bis(sulphosuccinimidyl) suberate (BS3 or BS³), glutaraldehyde or neuraminidase.

According to another embodiment, the liver targeting and/or the inhibition of the proinflammatory response is/are done by using a ionophore. By ionophore, it is meant as it is well known from the person skilled in the art a lipid-soluble molecule that allows the transport of ions across the lipid bilayer of the cell membrane. Ionophores may be in particular lipid-soluble molecules as synthesized by microorganisms to transport ions across the lipid bilayer of the cell membrane. Generally, the ionophore is able to form a complex with a ion and serves as ion-carrier.

In an embodiment, the ionophore is one forming a complex with a divalent cation such as calcium. According to the invention, the ionophore may be used with calcium, which induces an increase of the calcium intracellular concentration and an exposition of the phosphatidylserine, leading to an early aging of the red blood cells.

As an example, one may use the calcium ionophore A23187 (calcimycin). It is deemed that the ionophore such as A23187 induces a raise in intracellular calcium concentrations of the RBCs, leading to the senescence of the cells and that the phagocytosis of aged red blood cells represses the proinflammatory response. This is in accordance with Romero P. J., Romero E. A., Blood Cells Mol. Dis. 25 (1999) 9-19; and Bratosin D. et al., Cell Death Differ. 8 (2001) 1143-1156.

In a particular embodiment, at least two methods of targeting are combined, and, for example, the composition then comprises AP-containing red blood cells which are in the form of an immune complex and are chemically treated so as to promote their uptake in the liver, and phagocytosis by APCs, in particular by the KCs.

In an embodiment the red blood cells originate from the patient itself.

In another embodiment, the red blood cells originate from a blood-typing compatible donor.

The composition according to the invention may comprise one or more APs in the same red blood cells or each one in different red blood cells.

Techniques for encapsulating active ingredients in red blood cells are known and the basic technique by lysis-resealing, which is preferred herein, is described in patents EP-A-101 341 and EP-A-679 101, to which those skilled in the art may refer. According to this technique, the primary compartment of a dialysis element (for example, a dialysis tubing or a dialysis cartridge) is continuously fed with a suspension of red blood cells, while the secondary compartment contains an aqueous solution which is hypotonic with respect to the suspension of red blood cells, in order to lyse the red blood cells; next, in a resealing unit, the resealing of the red blood cells is induced in the presence of the AP by increasing the osmotic and/or oncotic pressure, and then a suspension of red blood cells containing the AP is collected.

Among the variants described up until now, preference is given to the method described in WO2006/016247, which makes it possible to efficiently, reproducibly, safely and stably encapsulate the AP. This method comprises the following steps:

1—suspension of a red blood cell pellet in an isotonic solution at a haematocrit level greater than or equal to 65%, cooling between +1 and +8° C.,

2—measurement of the osmotic fragility using a sample of red blood cells from said red blood cell pellet, it being possible for steps 1 and 2 to be carried out in any order (including in parallel),

3—lysis and internalization process of the AP, inside the same chamber, at a temperature constantly maintained between +1 and +8° C., comprising passing the suspension of red blood cells at a haematocrit level greater than or equal to 65%, and a hypotonic lysis solution cooled to between +1 and 8° C., through a dialysis cartridge; and the lysis parameters being adjusted according to the osmotic fragility previously measured; and

4—resealing process carried out in a second chamber, inside which the temperature is between +30 and +40° C., and in the presence of a hypertonic solution.

The “internalization” is intended to mean penetration of the AP inside the red blood cells.

In particular, for the dialysis, the red blood cell pellet is suspended in an isotonic solution at a high haematocrit level, greater than or equal to 65%, and preferably greater than or equal to 70%, and this suspension is cooled to between +1 and +8° C., preferably between +2 and 6° C., typically in the region of +4° C. According to a specific embodiment, the haematocrit level is between 65% and 80%, preferably between 70% and 80%.

The osmotic fragility is advantageously measured on the red blood cells just before the lysis step. The red blood cells or the suspension containing them are (is) advantageously at a temperature close to or identical to the temperature selected for the lysis. According to another advantageous feature of the invention, the osmotic fragility measurement is exploited rapidly, i.e. the lysis process is carried out shortly after the sample has been taken. Preferably, this period of time between taking the sample and beginning the lysis is less than or equal to 30 minutes, more preferably still less than or equal to 25, and even less than or equal to 20 minutes.

As regards the manner in which the lysis-resealing process is carried out, with the osmotic fragility being measured and taken into account, those skilled in the art may refer to WO2006/016247 for further details.

According to one feature of the invention, the composition according to the invention comprises, at the end, a suspension of red blood cells at a haematocrit level of between about 40% and about 70%, preferably between about 45% and about 55%, better still about 50%. It is preferably packaged in a volume of about 1 to about 250 ml. The packaging is preferably in a blood bag, syringe and the like, of a type suitable for blood transfusion or administration. The amount of encapsulated AP corresponding to the medical prescription is preferably entirely contained in the blood bag, syringe and the like.

An object of the invention is also a method for inducing, in a host, an immune tolerance to a peptidic or proteic active principle, said composition comprising red blood cells containing an active principle selected from the group consisting of a therapeutic peptide, polypeptide or protein, a peptidic or proteic autoantigen, a peptide, polypeptide or protein inducing an allergic reaction and a transplantation peptidic or proteic antigen. This method comprises the administration to the host of an effective amount of a composition according to the invention, in particular intravenously, by injection or infusion, preferably by infusion.

According to one feature of the invention, about 1 to about 250 ml, especially about 10 to about 250 ml, typically about 10 and about 200 ml of a suspension of red blood cells is administered. The suspension is at an appropriate haematocrit level, generally of between about 40% and about 70%, preferably between about 45% and about 55%, better still about 50%, are administered. The red blood cells may have their own tolerogenic effect with respect to the active principle that is presented at the same time (the encapsulated active principle). High amounts of red blood cells may thus favour the tolerogenic effect. On the other hand, targeting the liver as recited above may allow to use low doses of red blood cells. The person skilled in the art may thus select the optimal amount of active principle and of red blood cells used in a patient, and may take into account whether or not the red blood cells have been treated to target the liver.

An object of the invention is also the use of a composition according to the invention, for the induction of an immune tolerance specific to the active principle or the active principles that are present in the administered red blood cells.

Another object of the invention is a composition according to the invention, for use as a medicament to induce an immune tolerance specific to the active principle or the active principles that are present in the administered red blood cells.

The present invention will now be described in greater detail by means of embodiments taken by way of nonlimiting examples, and which refer to the attached drawings wherein:

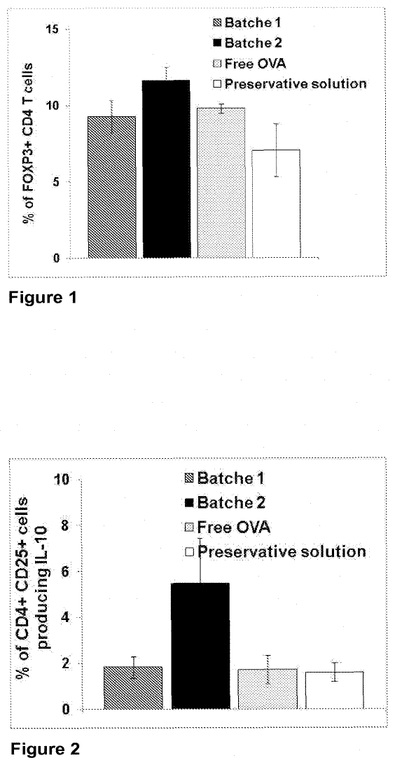

FIG. 1 is a graph representing the percentage of CD4 T cells expressing FOXP3

FIG. 2 is a graph representing the percentage of regulatory CD4⁺ CD25⁺ T cells producing IL-10.

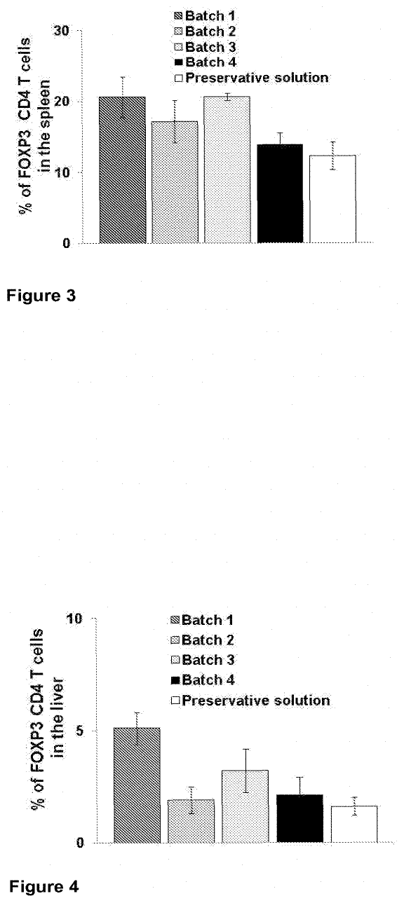

FIG. 3 is a graph representing the percentage of CD4 T cells expressing FOXP3 in the spleen.

FIG. 4 is a graph representing the percentage of CD4 T cells expressing FOX P3 in the liver.

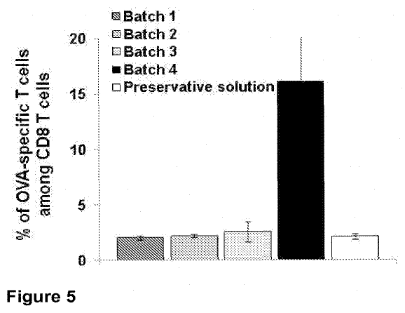

FIG. 5 is a graph representing the percentage of OVA-specific CD8 T cells.

### EXAMPLE 1: ENCAPSULATION OF FITC-DEXTRAN IN MURINE RED BLOOD CELLS

FITC-dextran fluorochrome (70 kDa) has been encapsulated in red blood cells of murine origin (OF1 mice) using the column hypotonic dialysis. Blood is centrifuged and then washed 3 times with PBS. Heamatocrit is adjusted to 70% in the presence of FITC-dextran added to a final concentration of 8 mg/ml before dialysis. The red blood cells are dialysed at a rate of 2 ml/min against a lysis tampon having a low osmolarity (counter-flux at 15 ml/min). The lysed red blood cells leaving the column are rescelled using a high osmolarity solution and incubation 30 min at 37° C. After several washings with PBS containing glucose, the cells are brought to heamatocrit 50%.

### EXAMPLE 2. CHEMICAL TREATMENT WITH BIS(SULPHOSUCCINIMIDYL) SUBERATE (BS3) 1 MM ON THE RED BLOOD CELLS CONTAINING FITC-DEXTRAN

The suspension of red blood cells encapsulating FITC-dextran is washed several times before being brought to 1.7×10⁶ cell/μl with PBS and mixed with one volume of a buffer solution of 2 mM BS3 (the BS3 solution contains glucose 0.09 and phosphate buffer, pH 7.4), so as to obtain a final BS3 concentration of 1 mM. The cells are incubated for 30 minutes at room temperature. The reaction is quenched by adding one volume of 20 mM Tris-HCl, NaCl 140 mM. After incubation at room temperature for 5 minutes, the mixture is centrifuged at 800 g for 5 min, 4° C. The cells are then washed twice with PBS containing glucose (centrifugation at 800 g) and once with SAG-BSA 6% (centrifugation at 1000 g) for 10 min, before adjustment to heamatocrit 50% to constitute the final products.

### EXAMPLE 3. CHEMICAL TREATMENT WITH BIS(SULPHOSUCCINIMIDYL) SUBERATE (BS3) 5 MM ON THE RED BLOOD CELLS CONTAINING FITC-DEXTRAN

The suspension of red blood cells encapsulating FITC-dextran is washed several times before being brought to 1.7×10⁶ cell/μ1 with PBS and mixed with one volume of a buffer solution of 10 mM BS3 (the BS3 solution contains glucose 0.09 and phosphate buffer, pH 7.4), so as to obtain a final BS3 concentration of 5 mM. The cells are incubated for 30 minutes at room temperature. The reaction is quenched by adding one volume of 20 mM Tris-HCl, NaCl 140 mM. After incubation at room temperature for 5 minutes, the mixture is centrifuged at 800 g for 5 min, 4° C. The cells are then washed twice with PBS containing glucose (centrifugation at 800 g) and once with SAG-BSA 6% (centrifugation at 1000 g) for 10 min, before adjustment to heamatocrit 50% to constitute the final products.

### EXAMPLE 4. TREATMENT OF THE RED BLOOD CELLS CONTAINING FITC-DEXTRAN BY THE A23187 IONOPHORE

The suspension of red blood cells containing FITC-dextran is washed once with a tampon A containing Hepes 10 mM, NaCl 140 mM, BSA 0.1%, CaCl₂ 2.5 mM, and then the suspension is diluted to 1.10⁶ cells/microliter using tampon A. Ionophore concentrated in DMSO is diluted with tampon A and then added to the cell suspension in order to get a final concentration of 0.15, 0.2 or 0.3 μM. The cells are incubated 30 min at 37° C. The mixture is centrifuged at 800 g during 6 min, 4° C. Then the cells are washed 2 times with PBS containing glucose (centrifugation 800 g) and once with SAG-BSA 6% (centrifugation 1000 g), and the final products are obtained.

### EXAMPLE 5. BIODISTRIBUTION OF FITC-DEXTRAN AFTER INJECTION OF THE RED BLOOD CELLS IN MICE

5 batches of red blood cells from mice OF1 containing FITC-dextran as obtained in example 1 and treated or not with BS3 or ionophore (based on examples 2-4) as follows were prepared:

Batch 1: no treatment

### Batch 2: BS3 1 mM

### Batch 3: BS3 5 mM

Batch 4: ionophore 0.2 μM
Batch 5: ionophore 0.3 μM.

Each batch is injected IV at J1 into OF1 mice. The mice are sacrificed 1 h30 after injection, and blood, spleen, liver and bone marrow are recovered: aliquots of 50 μl of blood and for spleen, liver and bone marrow aliquots of 50 μl after grinding and homogenisation of the whole cells of each organ. The aliquots are congelated for at least 20 min at −20° C., then thawn slowly at room temperature. The aliquots from the control mice are used to prepare a FITC-dextran standard range of concentration: the aliquots are then lysed with 125 μl of different concentrations of FITC-dextran to constitute the standard range of concentration.

The aliquots of the sample to be analysed are lysed using 125 μl of distilled water. Then 175 μl of TCA 12% are added to the aliquots. The mixtures are then centrifuged at 15,000 g, 10 min, 4° C. 200 μl of acid supernatant are taken and 500 μl of triethanolamine 0.4 M are added before fluorimetry detection (excitation at 494 nm, emission 521 nm). The FITC-dextran concentration of each sample can be determined using the standard range of concentration and the proportion of FITC-dextran present in the corresponding organ can then be deduced.

Biodistribution of FITC-Dextran 1 h30 after Injection of the Red Blood Cells in OF1 Mice (Table 1):

| Batches | Blood | Liver | Spleen |
|---|---|---|---|
| 1 | 57% | 19.8 ± 7.6% | 7.7 ± 1.9% |
| 2 | 35.5 ± 8% | 32 ± 4.6% | 22.8 ± 14% |
| 3 | 11% | 20.6% | 13.30% |
| 4 | 19.9 ± 12.6% | 24 ± 1.3% | 7.2 ± 1.2% |
| 5 | 14.7 ± 1.3% | 36.7 ± 7.2% | 9.2 ± 0.8% |

1 mM BS3 treatment induces erythrocyrte targeting of the liver and the spleen whereas ionophore treatment induces erythrocyrte targeting of the liver only. Increasing dose of ionophore enhances targeting.

### EXAMPLE 6. PHAGOCYTOSIS MEASUREMENT IN MICE OF FITC-DEXTRAN CONTAINING RED BLOOD CELLS

5 batches of red blood cells from mice OF1 containing FITC-dextran as obtained in example 1 and treated or not with BS3 or ionophore (based on examples 2-4) were prepared:

Batch 1: no treatment

### Batch 2: BS3 1 mM

### Batch 3: BS3 5 mM

Batch 4: ionophore 0.2 μM
Batch 5: ionophore 0.3 μM.

Each batch is injected IV at J1 into OF1 mice. The mice are sacrificed 1 h30 after injection, and livers are recovered. Fluorescence incorporated in the liver macrophages expressing F4/80 marker, the liver cells expressing CD11b marker and the liver dendritic cells expressing the CD11c marker were measures using flow cytometry.

Percentage of Liver Cells Having Phagocyted the FITC-Dextran Containing Red Blood Cells 1 h30 after Injection into Mice (Table 2):

| Batches | Macrophages F4/80 | CD 11b cells | Dendritic cells |
|---|---|---|---|
| 1 | 7.8% | 1.7% | 3.4% |
| 2 | 7.2% | 6.4% | 7.1% |
| 3 | 13.0% | 7.8% | 10.2% |
| 4 | 10.7% | 6.4% | 11.4% |
| 5 | 7.4% | 3.3% | 7.2% |

BS3 and ionophore treatments induce erythrophagocytosis by macrophages (F4/80 and CD 11b) and dendritic cells. For BS3 treatment, the percentage of cells that phagocyte treated red blood cells is dose dependent of the amount of BS3 used for treatment.

### EXAMPLE 7. METHOD FOR ENCAPSULATING OVALBUMIN IN MURINE AND HUMAN RED BLOOD CELLS

Variant 1:

Ovalbumin (protein of 45 kDa, hen egg ovalbumin) was encapsulated in murine red blood cells (OF1 mice or C57Bl/6 mice) by the method of hypotonic dialysis in dialysis tubing. The red blood cell suspension was washed several times before being brought to a haematocrit of 70% for the dialysis. The dialysis was carried out in dialysis tubing in a lysis buffer of low osmolarity for about 1 hour or 30 min when the dialysis occurred after a heat treatment. The red blood cells were then resealed by means of a solution of high osmolarity for 30 minutes. After a few washes, the final product was taken up in a buffer, Sag-mannitol, and haematocrit was brought to 50%.

Variant 2:

Ovalbumin was herein encapsulated in the murine red blood cells by the method of hypotonic dialysis in a dialysis column. The red blood cell suspension was washed several times before being brought to a haematocrit of 70% for the dialysis. The dialysis was carried out in a dialysis column in a lysis buffer of low osmolarity for about 10 min. As soon as they left the column, the red blood cells were resealed by means of a solution of high osmolarity for 30 minutes at 37° C. After a few washes, the final product was taken up in a NaCl glucose buffer containing glucose SAG mannitol, or decomplemented plasma, and haematocrit was brought back to 50%.

### EXAMPLE 8: METHOD FOR ENCAPSULATING OVALBUMIN IN MOUSE RED BLOOD CELLS

Ovalbumin (Worthington Biochemical Corporation, Lakewood, N.J.) was encapsulated into mouse red blood cells by hypotonic dialysis. Red blood cells suspensions were prepared from C57BL/6 mouse blood collected on lithium heparin. Briefly, the red blood cells were washed three times with saline solution and the haematocrit (Hct) of the blood was adjusted to 70% before dialysis. OVA were added to the red blood cells suspension at a final concentration of 5, or 0.5 mg/ml. Dialysis was performed (cell flow rate of 2 ml/min) against a cell lysis buffer (osmolality of 50 mOsmol/kg) circulating at counter-current (15 ml/min) into an 80 hollow-fiber dialyser (Gambro, Lyon, France). Red blood cells were resealed “on-line” by adding (10% final volume) an hypertonic solution (1900 mOsmol/kg) containing 0.4 g/l adenine (Sigma-Aldrich, Saint-Louis, Mich.), 15.6 g/l inosine (Sigma-Aldrich), 6.4 g/l sodium pyruvate (Sigma-Aldrich), 4.9 g/l monosodium phosphate dehydrate (Sigma-Aldrich), 10.9 g/l disodium phosphate dodecahydrate (Sigma-Aldrich), 11.5 g/l glucose monohydrate (Sigma-Aldrich) and 50 g/l NaCl (Sigma-Aldrich). Red blood cells were incubated 30 min at 37° C. with the hypertonic solution. Following several washings with 0.9% NaCl 0.2% glucose (Bioluz, Saint-Jean-de-Luz, France), the product was washed once with the tampon A containing Hepes 10 mM, NaCl 140 mM, BSA 0.1%, CaCl2 2.5 mM, diluted with tampon A to 1.10⁶ cells/μl and treated with 0.15 μM of Calcium ionophore A23187 (Sigma) for 30 min at 37° C. as described in the example 15. After 3 washes with 0.9% NaCl 0.2% glucose, the final product was resuspended and its haematocrit was adjusted to 50% with decomplemented C57BL/6 mouse plasma (15% final volume). The product thus obtained was stored at 2-8° C.

### EXAMPLE 9: METHOD FOR ENCAPSULATING OVALBUMIN IN MOUSE RED BLOOD CELLS

Ovalbumin (Worthington Biochemical Corporation, Lakewood, N.J.) was encapsulated into mouse red blood cells by hypotonic dialysis. Red blood cells suspensions were prepared from C57BL/6 mouse blood collected on lithium heparin. Briefly, the red blood cells were washed three times with saline solution and the haematocrit (Hct) of the blood was adjusted to 70% before dialysis. OVA were added to the red blood cells suspension at a final concentration of 5, or 0.5 mg/ml. Dialysis was performed (cell flow rate of 2 ml/min) against a cell lysis buffer (osmolality of 50 mOsmol/kg) circulating at counter-current (15 ml/min) into dialysis tubing. After dialysis, red blood cells were resealed by adding (10% final volume) an hypertonic solution (1900 mOsmol/kg) containing 0.4 g/l adenine (Sigma-Aldrich, Saint-Louis, Mich.), 15.6 g/l inosine (Sigma-Aldrich), 6.4 g/l sodium pyruvate (Sigma-Aldrich), 4.9 g/l monosodium phosphate dehydrate (Sigma-Aldrich), 10.9 g/l disodium phosphate dodecahydrate (Sigma-Aldrich), 11.5 g/l glucose monohydrate (Sigma-Aldrich) and 50 g/l NaCl (Sigma-Aldrich). Red blood cells were incubated 30 min at 37° C. with the hypertonic solution and then chemically treated with BS3 as described in the example 14.

Following several washings with 0.9% NaCl 0.2% glucose (Bioluz, Saint-Jean-de-Luz, France), the final product was resuspended and its haematocrit was adjusted to 50% with decomplemented C57BL/6 mouse plasma (15% final volume). The product thus obtained was stored at 2-8° C.

### EXAMPLE 10. ANTIBODY TREATMENT ON THE RED BLOOD CELLS CONTAINING OVALBUMIN

The suspension of red blood cells encapsulating ovalbumin is washed several times before being brought to 10⁹ cells/ml for the in vivo test and 10⁸ cell/ml for the in vitro test. It is incubated with the anti-TER119 antibody (10 μg/ml for the in vitro test and 23 μg/ml or 5 μg/ml for the in vivo test) for 30 minutes at 4° C. After a few washes, the final product is taken up in a buffer with injectable qualities, and haematocrit is brought to 50%.

### EXAMPLE 11. MEASUREMENT OF THE PHAGOCYTOSIS OF OVALBUMIN-CONTAINING RED BLOOD CELLS BY DENDRITIC CELLS IN VITRO

The effect of the antibody treatment on the phagocytosis efficiency of the red blood cells obtained according to example 9, by dendritic cells, is measured in vitro. The red blood cells are labelled with a fluorescent label, CFSE (carboxyfluorescein succinimidyl ester), for 20 min at 4° C. CFSE is a non-fluorescent dye which diffuses through the cell membrane. Once inside the cell, the molecule becomes fluorescent subsequent to its cleavage by intracellular esterases.

Dendritic cells are isolated from the spleen of C57131/6 mice using magnetic beads. These beads carry antibodies which recognize the CD11c marker, thereby making it possible to isolate the CD11c⁺ dendritic cell fraction.

The CFSE-labelled or unlabeled red blood cells are then incubated with the dendritic cells (10×10⁶ cell/ml) at a ratio of 20:1 in a final volume of 200 μl/well of round-bottomed 96-well culture plates for 4 hours at 37° C. and 5% CO₂. After culturing for 4 hours, the red blood cells not ingested by the dendritic cells are lysed with NH₄Cl, and several washes are carried out. The capture of the CFSE fluorochrome by the dendritic cells is then measured by flow cytometry (R. Segura et al., J. Immunol, January 2006, 176(1): 441-50).

Three populations of red blood cells were tested:

(A) red blood cells loaded with ovalbumin and not labelled with the CFSE fluorochrome,
(B) red blood cells loaded with ovalbumin and labelled with CFSE,
(C) red blood cells loaded with ovalbumin, treated with the anti-TER 119 antibody and labelled with CFSE.

Results

TABLE 3
Percentage of dendritic cells having phagocytosed
fluorescent red blood cells:

| Red blood cell population | % of dendritic cells |
|---|---|
| (A) | 4% |
| (B) | 27% |
| (C) | 36% |

The murine red blood cells loaded with ovalbumin and treated with the anti-TER 119 antibody were more efficiently phagocytosed by the dendritic cells isolated from the spleen than the untreated red blood cells in vitro, after 4 hours of coculture. 36% of the dendritic cells phagocytosed the red blood cells carrying the antibody, against only 27% in the absence of antibody.

### EXAMPLE 12. MEASUREMENT OF THE PHAGOCYTOSIS OF RED BLOOD CELLS CONTAINING OVALBUMIN, BY MACROPHAGES AND DENDRITIC CELLS OF THE SPLEEN AND LIVER IN VIVO ON MICE

This study is an allogenic study since OF1 mice red blood cells containing ovalbumin are injected to not consanguineous C57Bl/6 mice.

Three batches of 74×10⁷ red blood cells, from OF1 mice, loaded with ovalbumin (example 9) treated with the anti-TER 119 antibody (as described in example 10) or not treated are prepared. These batches are divided up in the following way:

Batch 1: no antibody treatment
Batch 2: treated with the anti-TER 119 antibody.

Each batch is labelled with CFSE and injected intravenously into C57Bl/6 mice. Three hours after the injection, the blood, the spleen and the liver of the mice are taken. The percentage of fluorescent red blood cells circulating in the blood of the mice is measured by flow cytometry. The fluorescence incorporated into the spleen macrophages expressing the F4/80 marker, into the liver macrophages expressing the F4/80 marker and into the spleen dendritic cells expressing the CD11c marker is measured by flow cytometry.

Results

TABLE 4
Percentage of macrophages or dendritic cells from
the spleen, having phagocytosed fluorescent red blood
cells 3 hours after injection into the mouse:

| Batches | Macrophages | Dendritic cells |
|---|---|---|
| 1 | 28% | 5% |
| 2 | 81% | 22% |

3 hours after injection, the murine red blood cells loaded with ovalbumin and treated with the anti-TER 119 antibody are almost no longer present in the blood of the mouse (1%), whereas there are still untreated, ovalbumin-loaded red blood cells in the blood of the mouse (4.6%).

The red blood cells that have been treated with the anti-TER 119 antibody are phagocytosed by the F4/80 macrophages and CD11c dendritic cells of the spleen.

The red blood cells treated with the anti-TER 119 antibody were more efficiently phagocytosed by the F4/80 macrophages of the spleen than the untreated red blood cells. 81% of the spleen macrophages phagocytosed the antibody-treated red blood cells, against only 28% in the untreated batch (Table 4).

The antibody-treated red blood cells were also more efficiently phagocytosed by the CD11c dendritic cells from the spleen than the untreated red blood cells. Respectively 22% of dendritic cells phagocytosed the antibody-treated red blood cells against only 5% in the case of the untreated red blood cells (Table 4).

TABLE 5
Percentage of liver macrophages having phagocytosed fluorescent
red blood cells 3 hours after injection into the mouse.

| Batches | Macrophages |
|---|---|
| 1 | 24% |
| 2 | 50% |

The red blood cells treated with the anti-TER 119 antibody are phagocytosed by the F4/80 macrophages of the liver.

The red blood cells treated with the anti-TER 119 antibody were more efficiently phagocytosed by the F4/80 macrophages of the liver than the untreated red blood cells. 50% of the liver macrophages phagocytosed the antibody-treated red blood cells, against only 24% in the untreated batch (table 5).

In conclusion, the binding of the antibody to the red blood cells allowed efficient targeting of the red blood cells in the spleen and the liver, and a significant increase in the percentage of dendritic cells and of macrophages capable of phagocytizing these red blood cells.

### EXAMPLE 13. MEASURE OF PERCENTAGE OF REGULATORY T CELLS AND THEIR PRODUCTION OF ANTI-INFLAMMATORY INTERLEUKIN-10 (IL-10) AFTER ONE INJECTION OF POLY(I:C) AND ANTIBODY-TREATED OR UNTREATED OVALBUMIN-LOADED ERYTHROCYTES IN MICE

The purpose of this study was to measure the percentage of regulatory T cells in C57Bl/6 mice after injection of Poly(I:C) and antibody-treated (anti-TER 119) or untreated ovalbumin-loaded erythrocytes.

Two batches of 30×10⁷ antibody-treated or untreated ovalbumin-loaded erythrocytes from OF1 mice were prepared according to example 8. In this study, an equivalent amount of entrapped ovalbumin was injected free and the negative control was the preservative solution of erythrocytes (NaCl glucosed containing 33% of decomplemented mice plasma). The amount of free or entrapped OVA injected to mice was 2 μg. The amount of free Poly(I:C) injected to mice was 25 μg.

Batch 1: ovalbumin-loaded erythrocytes and Poly(I:C)
Batch 2: antibody-treated ovalbumin-loaded erythrocytes and Poly(I:C)

The batches were injected intravenously to C57Bl/6 mice (4 mice per group). Seven days after batch injection, mice were killed and their spleens collected. To measure the percentage of FOXP3 expressing CD4⁺ T cells by flow cytometry (FIG. 1, Table 6), 2.5×10⁶ of spleen cells were used. Briefly, after RBC lysis using NH₄Cl solution (StemCell Technologies, catalogue number 7850), spleen cells were first stained with PC5-anti-CD4 (Biolegend, catalogue number BLE100514) and FITC-anti-CD25 monoclonal antibodies (Biolegend, catalogue number BLE110569), and then incubated with fixation and permeabilisation buffers (Biolegend, catalogue number 421303) before being incubated with PE-anti-FOXP3 mAb (Biolegend, catalogue number 320008) or isotype control.

To measure the percentage of regulatory T cells (CD4+CD25+) producing IL-10 by flow cytometry (FIG. 2), spleen cells (5×10⁶ cells/ml) were cultured with 0.1 μg/ml of OVA323-339 peptide (Genscript, catalogue number 41007-1) for 4 hours at 37° C. in 5% CO₂-air on 24-well culture plates. One hour after the beginning of the culture, Brefeldin A (Ebioscience, catalogue number 420601) were added to block cytokine secretion. At the end of the culture, cells were first stained with PC5-anti-CD4 and FITC-anti-CD25 monoclonal antibodies, and then incubated with fixation buffer (Biolegend, catalogue number 420801), and permeabilisation buffer (Biolegend, catalogue number 421002) before being incubated with PE-anti-IL-10 mAb (Biolegend, catalogue number 505008) or isotype control.

The percentage of regulatory CD4 T cells expressing the transcription factor FOXP3 had significantly increased after injection of Poly(I:C) and antibody-treated OVA-loaded erythrocytes or free OVA compared to control mice injected with the preservative solution (FIG. 1, Table 6, student test, p<0.007 and p<0.05 respectively).

TABLE 6
Percentage of CD4 T cells expressing FOXP3

| | % of CD4 T cells |
|---|---|
| | expressing FOXP3 |
| ITEMS | (±standard deviation) |
| Batch 1 | 9.3 ± 1.1 |
| Batch 2 | 11.6 ± 0.9 |
| Free OVA and Poly(I:C) | 8.6 ± 1.5 |
| Preservative solution | 7.0 ± 1.7 |

Nevertheless, only regulatory T cells induced by antibody-treated OVA-loaded erythrocyte and Poly(I:C) injection can produce the anti-inflammatory cytokine (IL-10) after in vitro restimulation with OVA peptide (FIG. 2, table 7).

TABLE 7
Percentage of regulatory CD4⁺ CD25⁺ T cells producing IL-10

| | % of CD4 CD25 cells |
|---|---|
| | producing IL-10 |
| ITEMS | (±standard deviation) |
| Batch 1 | 1.8 ± 0.5 |
| Batch 2 | 5.5 ± 2 |
| Free OVA and Poly(I:C) | 1.7 ± 0.6 |
| Preservative solution | 1.6 ± 0.4 |

In summary, injection of antibody-treated OVA-loaded erythrocyte and Poly(I:C) induced the generation of regulatory T cells able to produce IL-10 after restimulation with antigen.

In FIG. 1, the percentage of FOXP3+CD4⁺ T cells in the spleen was determined by flow cytometry 7 days after intravenous injection into C57BL/6 mice of antibody-treated (black bars) or untreated (dark grey bars) OVA-loaded erythrocytes and Poly(I:C) or free OVA and Poly(I:C) (light grey bars), or control medium (white bars). The amount of free or entrapped OVA injected to mice was 2 μg and the amount of and Poly(I:C) was 25 μg.

In FIG. 2, the production of IL-10 by regulatory CD4⁺ CD25+ T cells was determined by flow cytometry after in vitro restimulation with OVA peptide 0.1 μg/ml of spleen cells isolated from C57BL/6 mice injected 7 days with antibody-treated (black bars) or untreated (dark grey bars) OVA-loaded erythrocytes and Poly(I:C) or free OVA (light grey bars) and Poly(I:C), or control medium (white bars). The amount of free or entrapped OVA injected to mice was 2 μg and the amount of and Poly(I:C) was 25 μg.

### EXAMPLE 14. BS3 TREATMENT ON THE RED BLOOD CELLS CONTAINING OVALBUMIN

The suspension of red blood cells encapsulating OVA (example 8) was washed several times before being brought to 1.7×10⁶ cells/μl with PBS and mixed with one volume of a buffer solution of 2 mM BS3 (the BS3 solution contained glucose 0.09% and phosphate buffer, pH7.4), so as to obtain a final BS3 concentration of 1 mM. The cells were incubated for 30 minutes at room temperature. The reaction was quenched by adding one volume of 20 mM Tris-HCl, NaCl 140 mM. After incubation at room temperature for 5 minutes, the mixture was centrifugated at 800 g for 5 min, 4° C. The cells were then washed thrice with NaCl glucose (centrifugation at 800 g) for 10 min, before adjustment to hematocrit 50% with decomplemented plasma.

### EXAMPLE 15. IONOPHORE TREATMENT ON THE RED BLOOD CELLS CONTAINING OVALBUMIN

The suspension of red blood cells encapsulating OVA (example 8) was washed once with a tampon A containing Hepes 10 mM, NaCl 140 mM, BSA 0.1%, CaCl₂) 2.5 mM, and then the suspension was diluted to 1.10⁶ cells/μl using tampon A. Ionophore concentrated in DMSO was diluted with tampon A and then added to the cell suspension in order to get a final concentration of 0.15 μM. The cells were incubated 30 min at 37° C. The mixture was centrifugated at 800 g during 6 min, 4° C. The cells were then washed thrice with NaCl glucose (centrifugation at 800 g) for 10 min, before adjustment to hematocrit 50% with decomplemented plasma.

### EXAMPLE 16. MEASURE OF PERCENTAGE OF REGULATORY T CELLS IN THE LIVER AND IN THE

spleen after one injection of ovalbumin-loaded erythrocytes in mice treated to target the liver and/or to repress APC proinflammation response

The purpose of this study was to demonstrate that the use of RBC, treated to target the liver and to repress APC proinflammation response, as antigen delivery system induced an increase in the percentage of regulatory T cells in mice.

Batches of 126×10⁷ antibody-treated, BS3-treated or ionophore-treated ovalbumin-loaded erythrocytes from C57BL/6 mice were prepared according to example 8. The amount of entrapped OVA injected to mice was 8 μg.

Batch 1: ionophore-treated ovalbumin-loaded erythrocytes
Batch 2: BS3-treated ovalbumin-loaded erythrocytes
Batch 3: antibody-treated ovalbumin-loaded erythrocytes
Batch 4: antibody-treated ovalbumin-loaded erythrocytes and Poly(I:C)

The batches were injected intravenously to C57Bl/6 mice (3 mice per group). Seven days after batch injection, mice were killed and their spleens and liver collected. To measure the percentage of FOXP3 expressing CD4⁺ T cells by flow cytometry in the spleen (FIG. 3, Table 8) and in the liver (FIG. 4, Table 8), 1×10⁶ and 2.5×10⁶ of liver cells and spleen cells were used. Briefly, after RBC lysis using NH4Cl solution (StemCell Technologies, cat nb 7850), cells were first stained with PC5-anti-CD4 (Biolegend, cat nb BLE100514) and FITC-anti-CD25 monoclonal antibodies (Biolegend, cat nb BLE110569), and then incubated with fixation and permeabilisation buffers (Biolegend, cat nb 421303) before being incubated with PE-anti-FOXP3 mAb (Biolegend, cat nb 320008) or isotype control.

In FIG. 3, the percentage of FOXP3+CD4+ T cells in the spleen was determined by flow cytometry 7 days after intravenous injection into C57BL/6 mice of ionophore-treated (dark grey bar), BS3-treated (grey bar) or antibody-treated (light grey bar) OVA-loaded erythrocytes or antibody-treated OVA-loaded erythrocytes and Poly(I:C) (black bar) or control medium (white bar). The amount of OVA entrapped injected to mice was 8 μg.

In FIG. 4, the percentage of FOXP3+CD4+ T cells in the liver was determined by flow cytometry 7 days after intravenous injection into C57BL/6 mice of ionophore-treated (dark grey bar), BS3-treated (grey bar) or antibody-treated (light grey bar) OVA-loaded erythrocytes or antibody-treated OVA-loaded erythrocytes and Poly(I:C) (black bar) or control medium (white bar). The amount of OVA entrapped injected to mice was 8 μg.

To measure the percentage of OVA-specific CD8 T cells by flow cytometry (FIG. 5, Table 9), spleen cells were stained with PC7-anti-CD8 (Biolegend, cat nb BLE100722), FITC-anti-CD3 (Biolegend, cat nb BLE100203) and PC5-anti-CD62L monoclonal antibodies (Biolegend, cat nb BLE104410) and PE-OVA-tetramer (Beckman Coulter, cat nb T20076).

In FIG. 5, the percentage of OVA-specific CD8+ T cells in the spleen was determined by flow cytometry 7 days after intravenous injection into C57BL/6 mice of ionophore-treated (dark grey bar), BS3-treated (grey bar) or antibody-treated (light grey bar) OVA-loaded erythrocytes or antibody-treated OVA-loaded erythrocytes and Poly(I:C) (black bar) or control medium (white bar). The amount of OVA entrapped injected to mice was 8 μg.

To measure the percentage of regulatory T cells (CD4+CD25+) producing IL-10 by flow cytometry (Table 10), spleen cells (5×10⁶ cells/ml) were cultured with 0.1 μg/ml of OVA323-339 peptide (Genscript, cat nub 41007-1) or without peptide for 4 hours at 37° C. in 5% CO₂-air on 24-well culture plates. One hour after the beginning of the culture, Brefeldin A (Ebioscience, cat nb 420601) were added to block cytokine secretion. At the end of the culture, cells were first stained with PC5-anti-CD4 and FITC-anti-CD25 monoclonal antibodies, and then incubated with fixation buffer (Biolegend, cat nb 420801), and permeabilisation buffer (Biolegend, cat nb 421002) before being incubated with PE-anti-IL-10 mAb (Biolegend, cat nb 505008) or isotype control.

The ionophore treatment was the only treatment able to induce a significant increase of regulatory CD4 T cells expressing FOXP3 in both the spleen and the liver (Table 8 and, FIGS. 3 and 4, p<0.05). The antibody treatment was able to induce a significant increase of regulatory CD4 T cells expressing FOXP3 in the spleen only (Table 8, FIG. 3, p<0.05).

TABLE 8
Percentage of regulatory FOXP3
CD4⁺ T cells in the spleen and the liver

| | % of CD4 expressing | % of CD4 expressing |
|---|---|---|
| | FOXP3 in the spleen | FOXP3 in the liver |
| ITEMS | (±standard deviation) | (±standard deviation) |
| Batch 1 | 20 ± 3 | 5 ± 1 |
| Batch 2 | 17 ± 3 | 2 ± 1 |
| Batch 3 | 21 ± 0 | 3 ± 1 |
| Batch 4 | 14 ± 2 | 2 ± 1 |
| Preservative solution | 12 ± 2 | 2 ± 0 |

Only the co-injection of Ab-treated OVA-loaded RBC and Poly(I:C) induced an increase in the percentage of OVA-specific CD8 T cells (Table 9, FIG. 5).

TABLE 9
Percentage of OVA-specific CD8⁺ T cells in the spleen

| | % of OVA-specific CD8 T cells |
|---|---|
| ITEMS | (±standard deviation) |
| Batch 1 | 2 ± 0 |
| Batch 2 | 2 ± 0 |
| Batch 3 | 3 ± 1 |
| Batch 4 | 16 ± 7 |
| Preservative solution | 2 ± 0 |

Only the co-injection of Ab-treated OVA-loaded RBC and Poly(I:C) induced an increase in the percentage of CD4⁺ CD25⁺ T producing IL-10 in the spleen and this production was not specific to OVA (Table 10).

TABLE 10
Percentage of regulatory CD4⁺ CD25⁺
T producing IL-10 in the spleen

| | Restimulation with | Restimulation without |
|---|---|---|
| | OVA peptide | OVA peptide |
| ITEMS | (±standard deviation) | (±standard deviation) |
| Batch 1 | 5 ± 1 | 6 ± 1 |
| Batch 2 | 4 ± 0 | 7 ± 7 |
| Batch 3 | 7 ± 6 | 9 ± 1 |
| Batch 4 | 16 ± 5 | 21 ± 7 |
| Preservative solution | 4 ± 1 | 7 ± 1 |

### EXAMPLE 17. MEASURE OF IMMUNE TOLERANCE TO OVA AFTER THREE INJECTIONS OF OVALBUMIN-LOADED ERYTHROCYTES IN MICE TREATED TO TARGET THE LIVER AND REPRESS APC PROINFLAMMATION RESPONSE

The purpose of this study was to demonstrate that injections of OVA-loaded RBC, treated to target the liver and repress APC proinflammation response, inhibited the OVA T and B cell responses induced by OVA and Poly(I:C).

Before treatment, samples of blood (200 μl) from the C57BL/6 mice were collected by retro-orbital puncture on serum gel separator tube (Becton Dickinson, Microtainer TM SST, ref 365951) to obtain the pre-immun sera. Mice were then injected intravenously thrice at days −7, −3 and −1 with control medium, free OVA or batch of ionophore-treated ovalbumin-loaded erythrocytes prepared with blood from C57BL/6 mice and according to example 8 (3 to 4 mice per group). The amount of free and entrapped OVA injected to mice was 120 and 90 μg, respectively. At day 0 and 21, the mice were challenged with OVA and Poly(I:C) (100 μg and 50 μg/mice, respectively). Some mice received the control medium only. Six days after the last injection, samples of blood (200 μl) from the mice were collected to obtain the post-immun sera and a suspension of CFSE-labelled C57BL/6 splenocytes presenting or not the OVA257-264 peptide at a 1:1 ratio were injected to the mice to evaluate the capacity of cytotoxic CD8 T cells to lyse SIINFEKL cells. 16 hours after the injection of OVA257-264 cells, the mice were killed and their spleens collected.

To measure the percentage of activated and OVA-specific CD8 T cells by flow cytometry (Table 11), spleen cells were stained with PC7-anti-CD8 (Biolegend, cat nb BLE100722), FITC-anti-CD3 (Biolegend, cat nb BLE100203) and PC5-anti-CD62L monoclonal antibodies (Biolegend, cat nb BLE104410) and PE-OVA-tetramer (Beckman Coulter, cat nb T20076).

To measure the percentage of OVA-specific in vivo lysis (Table 12), the percentage of CFSElow and CFSEhigh cells were measured by flow cytometry and determined by the following formula:

%=[1−(ratio of treated mice/ratio of untreated mice)]×100, with ratio=percentage of CFSEhigh/percentage of CFSElow

To measure the anti-OVA IgG1 and IgG2a titer in the sera (Table 13), various dilutions of the pre- and post-immun sera (from 1/50 to 1/36450) were incubated in a 96-well MaxiSorp plates (Nunc, cat nb 442404) pre-coated with OVA (Serlabo, cat nb WQ-LS003054, 5 μg/ml). The presence of anti-OVA IgG1 and IgG2a was revealed by incubation of HorseRadishPeroxidase (HRP)-conjugated to anti-mouse IgG1 (Thermo Scientific, cat nb cat PA1-86031, dilution 1/4000) or anti-mouse IgG2a (Thermo Scientific, cat nb cat PA1-86039, dilution 1/4000) followed by the tetramethylbenzidine (TMB) substrate (Biolegend, cat nb 421101) incubation. The reaction was stopped by a solution of 2N H2SO4 and optical density was measured at 450 nm and 630 nm using a plate reader (Biotek, cat nb ELx808). Data obtained at 630 nm were subtracted to the data obtained at 450 nm and the antibody titer was determined as the dilution for which the optical density is higher than 3 times the O.D. obtained for the pre-immun sera diluted at 1/50.

The injections of ionophore-treated OVA-loaded RBC were able to significantly reduce the proliferation and activation of OVA-specific CD8 T cells induced by OVA and Poly(I:C) compared to the injections of OVA (Table 11; p<0.05).

TABLE 11
Percentage of activated and OVA-specific
CD8⁺ T cells in the spleen

| | | % of activated |
|---|---|---|
| | % of OVA-specific | OVA-specific |
| | CD8 T cells | CD8 T cells |
| Mice treatment | (±standard deviation) | (±standard deviation) |
| 3x Batch + | 1.5 ± 0.1 | 19 ± 9 |
| 2xOVA and Poly(I:C) | | |
| 3x OVA + | 2.1 ± 0.9 | 45 ± 15 |
| 2xOVA and Poly(I:C) | | |
| 2x OVA and Poly(I:C) | 4.8 ± 1.3 | 76 ± 7 |
| Preservative solution | 1.1 ± 0.1 | 6 ± 3 |

The injections of ionophore-treated OVA-loaded RBC were able to significantly reduce the OVA-specific cellular lysis induced by OVA and Poly(I:C) (Table 12; p<0.01).

TABLE 12
Percentage of OVA-specific in vivo lysis

| | % of OVA-specific in vivo lysis |
|---|---|
| Mice treatment | (±standard deviation) |
| 3x Batch + | 61 ± 15 |
| 2xOVA and Poly(I:C) | |
| 3x OVA + | 73 ± 19 |
| 2xOVA and Poly(I:C) | |
| 2 x OVA and Poly(I:C) | 98 ± 1 |
| Preservative solution | 1 ± 2 |

Mice pretreated with ionophore-treated OVA-loaded RBC had very low and/or no anti-OVA IgG1 and IgG2a antibody titers compared to mice pretreated with OVA (Table 13).

TABLE 13
Anti-OVA IgG1 and IgG2a antibody titer in the sera

| | Results for each mice | |
|---|---|---|
| Mice treatment | Anti-OVA IgG1 titer | Anti-OVA IgG2a titer |
| 3x Batch + | 0 | 0 |
| 2xOVA and Poly(I:C) | 900 | 0 |
| | 100 | 0 |
| | 100 | 0 |
| 3x OVA + | 12150 | 2700 |
| 2xOVA and Poly(I:C) | 20250 | 450 |
| | 4050 | 900 |
| | 300 | 0 |
| 2 x OVA and Poly(I:C) | 12150 | 8100 |
| | 1350 | 0 |
| | 150 | 150 |
| Preservative solution | 0 | 0 |
| | 0 | 0 |
| | 0 | 0 |
| | 0 | 0 |

### EXAMPLE 18. MEASURE OF IMMUNE TOLERANCE TO OVA AFTER INJECTIONS OF VARIOUS QUANTITIES OF OVALBUMIN-LOADED ERYTHROCYTES IN MICE

The experiment was performed as described in example 17, except that 2 different batches of ionophore-treated ovalbumin-loaded erythrocytes were prepared with 2 different concentrations of OVA, leading to one batch, batch 1, with 53250±6800 OVA molecules per RBC as in example 17 and another batch, batch 2, with 8250±840 molecules per RBC.

C57Bl/6 mice were injected intravenously thrice at days −7, −3 and −1 with 110 μl or 30 μl of batch 1, 110 μl of batch 2, 110 μl of free OVA or 110 μl of preservative solution. The amount of OVA and RBC injected to mice are presented in Table 14. At day 0 and day 21, the mice were challenged with OVA and Poly(I:C) (100 μg and 50 μg/mice, respectively). Six days after the last injection, the mice were killed, the spleen collected and the quantity of IgG1, IgG2b and IgG2c was measured in the sera as described in example 17 using HRP-conjugated to anti-mouse IgG2b (Southern Biotech, 1090-05) and anti-mouse IgG2c (Southern Biotech, 1079-05). To measure IFNγ production, spleen cells (5×10⁶ cells/ml) were first cultured with 0.1 μg/ml of OVA323-339 peptide (Genscript, cat nub 41007-1) or without peptide for 48 hours at 37° C. in 5% CO₂-air on 24-well culture plates. Then, IFNγ was measured in the supernatant by flow cytometry using Cytometric Bead Array (BD Bioscience, 558296 and 558266). To measure the percentage of cytotoxic T-Lymphocyte Antigen 4 (CTLA-4) expressing CD4+CD25+ T cells by flow cytometry, spleen cells were first stained with PC5-anti-CD4 (Biolegend, cat nb BLE100514) and FITC-anti-CD25 monoclonal antibodies (Biolegend, cat nb BLE110569), and then fixed with PBS 1% paraformaldehyde (Sigma Aldrich, F1635-25 ml) and permeabilized with saponin 0.3% (Sigma Aldrich, 84510) before being incubated with PE-anti-CTLA-4 mAb (BD Pharmingen, cat nb 553720) or isotype control.

TABLE 14
Quantities of OVA and RBC injected to mice at each injection

| | Mice treatment | | | | | |
|---|---|---|---|---|---|---|
| | OVA | | | OVA-loaded RBC | | |
| | (μg/mice) | | | (×10⁹ RBC/mice) | | |
| Injection number | 1 | 2 | 3 | 1 | 2 | 3 |
| Batch 1 High dose | 99 | 86 | 86 | 1.7 | 1.5 | 1.5 |
| (110 μl/mice) | | | | | | |
| Batch 1 Low dose | 24 | 23 | 23 | 0.4 | 0.4 | 0.4 |
| (30 μl/mice) | | | | | | |
| Batch 2 (110 μl/mice) | 11 | 8 | 11 | 1.6 | 1.6 | 1.6 |
| OVA (110 μl/mice) | 110 | 110 | 113 | | | |

Mice pretreated with Batch 1 High dose, high quantity of OVA and RBC, had significantly lower anti-OVA IgG1, IgG2b and IgG2c antibody titers than mice pretreated with OVA (Table 15, IgG1: p<0.002, IgG2b and IgG2c: p 0.05). Moreover, mice pretreated with Batch 2, same number of RBC but lower dose of OVA (8 to 11 μg/mice), had also significantly lower anti-OVA IgG1 and IgG2c antibody titers than mice pretreated with OVA (Table 15, IgG1: p<0.006, and IgG2c: p≤0.05). However, mice pretreated with Batch 1 Low dose, small quantity of OVA and RBC, had significant antibody titers. Thus, the quantity of OVA and RBC injected per mice plays a key role in immune tolerance induction.

TABLE 15
Anti-OVA IgG1, IgG2b and IgG2c antibody titer in the sera

| | IgG titer for each mice | | |
|---|---|---|---|
| | Anti-OVA | Anti-OVA | Anti-OVA |
| Mice treatment | IgG1 titer | IgG2b titer | IgG2c titer |
| 3x Batch1 High dose + | 900 | 8100 | 2700 |
| 2xOVA and Poly(I:C) | 4050 | 450 | 2700 |
| | 50 | 300 | 100 |
| | 12150 | 900 | 1350 |
| 3x Batch1 Low dose + | 36450 | 2700 | 24300 |
| 2xOVA and Poly(I:C) | 12150 | 12150 | 24300 |
| | 12150 | 2700 | 36450 |
| 3x Batch2 + | 0 | 0 | 300 |
| 2xOVA and Poly(I:C) | 2700 | 24300 | 8100 |
| | 8100 | 4050 | 900 |
| 3x OVA + | 36450 | 36450 | 36450 |
| 2xOVA and Poly(I:C) | 36450 | 2700 | 2700 |
| | 36450 | 36450 | 36450 |
| | 36450 | 36450 | 36450 |
| 2 x OVA and Poly(I:C) | 36450 | 2700 | 4050 |
| | 12150 | 8100 | 12150 |
| | 1350 | 12150 | 12150 |
| | 8100 | 24300 | 36450 |
| Preservative solution | 0 | 0 | 0 |
| | 0 | 0 | 0 |
| | 0 | 0 | 0 |
| | 0 | 0 | 0 |

The challenge OVA and Poly(I:C) induced an increase of IFNγ production in response to OVA stimulation. This increase was observed in the mice pretreated with free OVA but not with mice pretreated with ionophore-treated OVA-loaded RBC Table 16, (p<0.005).

TABLE 16
IFNγ production by splenocytes stimulated with OVA class
2-restricted peptide (average ± standard deviation)

| Mice treatment | IFNγ□(pg/ml) |
|---|---|
| 3x Batch1 High dose + | 43 ± 6 |
| 2xOVA and Poly(I:C) | |
| 3x Batch1 Low dose + | 38 ± 8 |
| 2xOVA and Poly(I:C) | |
| 3x Batch2 + | 41 ± 10 |
| 2xOVA and Poly(I:C) | |
| 3x OVA + | 82 ± 9 |
| 2xOVA and Poly(I:C) | |
| 2 x OVA and Poly(I:C) | 90 ± 36 |
| Preservative solution | 47 ± 25 |

As CTLA-4 is a protein which plays an important regulatory role in immune system by transmission of an inhibitory signal to T cells, its expression was measured on CD4 CD25 regulatory T cells by flow cytometry. Mice pretreated with Batch 1 High dose and Batch 2 had significantly higher percentage of CTLA-4 expression in CD4 CD25 regulatory T cells than mice pretreated with OVA and mice which have received the OVA+ Poly(I:C) challenge only (Table 17, Batch 1: p≤0.03 and p≤0.02, respectively and Batch 2: p≤0.05). Moreover, mice pretreated with Batch 1 High dose had also significantly higher mean fluorescence intensity (MFI) of CTLA-4 expression in CD4 CD25 regulatory T cells (Table 17, p≤0.03). Finally, mice pretreated with Batch 1 Low dose had significantly higher percentage of CTLA-4 expression in CD4 CD25 regulatory T cells than mice which have received the OVA+ Poly(I:C) challenge only (Table 17, p≤0.04).

TABLE 17
Percentage and mean fluorescence intensity (MFI) of CTLA-4 in
CD4 CD25 regulatory T cells (average ± standard deviation)

| | % of CTLA-4+ cells | MFI of CTLA-4 |
|---|---|---|
| Mice treatment | in CD4 CD25 T cells | in CD4 CD25 T cells |
| 3x Batch1 High dose + | 38 ± 3 | 361 ± 12 |
| 2xOVA and Poly(I:C) | | |
| 3x Batch1 Low dose + | 38 ± 3 | 363 ± 13 |
| 2xOVA and Poly(I:C) | | |
| 3x Batch2 + | 37 ± 2 | 357 ± 10 |
| 2xOVA and Poly(I:C) | | |
| 3x OVA + | 32 ± 3 | 341 ± 15 |
| 2xOVA and Poly(I:C) | | |
| 2 x OVA and Poly(I:C) | 31 ± 3 | 341 ± 6 |
| Preservative solution | 34 ± 6 | 348 ± 26 |

In conclusion, treatment with ionophore-treated antigen-loaded RBC allows preventing or reducing antigen-specific T and B cell response in a preventive model. Not only the quantity of antigen, but also the quantity of RBC injected per mice plays a key role in this therapy.

1-38. (canceled)

39. A method for inducing immune tolerance in a human subject, the method comprising:
administering, to a human subject in need thereof, an effective amount of an active principle to thereby induce immune tolerance to the active principle in the human subject,
wherein the active principle is encapsulated in an erythrocyte comprising a modification that promotes targeting of the erythrocyte to the reticuloendothelial system of the liver and/or spleen,
wherein the modification is formed by chemically treating the erythrocyte with an ionophore.

40. The method of claim 39, wherein the ionophore comprises calcimycin.

41. The method of claim 39, wherein the modification comprises an exposition of phosphatidylserine.

42. The method of claim 39, wherein the modification comprises an increased concentration of intracellular calcium as compared to an unmodified erythrocyte.

43. The method of claim 39, comprising administering from 1 to 250 mL of a suspension comprising the erythrocyte.

44. The method of claim 43, wherein the suspension comprises a haemocrit level of from about 40% to about 70%.

45. The method of claim 39, wherein the method results in an increase in CD4 T cells expressing FOXP3 in the spleen and liver.

46. The method of claim 39, wherein the method results in an increase in regulatory CD4⁺ CD25⁺ T cells that produce IL-10.

47. The method of claim 39, wherein the active principle comprises a peptide.

48. The method of claim 39, wherein the active principle comprises a protein.

49. A method for inducing immune tolerance in a human subject, the method comprising:
administering, to a human subject in need thereof, an effective amount of an active principle to thereby induce immune tolerance to the active principle in the human subject,
wherein the active principle is encapsulated in an erythrocyte comprising a modification that promotes targeting of the erythrocyte to the reticuloendothelial system of the liver and/or spleen, wherein the modification is formed by chemically treating the erythrocyte with an ionophore, and
wherein polyinosinic:polycytidylic acid (Poly(I:C)) is not administered to the subject.
